(19) United States
(12) Patent Application Publication
(10) Pub. No.: US 2023/0012903 A1
(43) Pub. Date: Jan. 19, 2023

(54) DEVICES, SYSTEMS, AND METHODS FOR RESPIRATORY DISEASE TESTING CROSS-REFERENCE TO RELATED APPLICATIONS

(71) Applicant: SiPhox, Inc., Burlington, MA (US)

(72) Inventors: Yulia Rybakova, Somerville, MA (US); Cole Chapman, Billerica, MA (US); Gordon Siek, Cambridge, MA (US); Jordan Cobb, Cambridge, MA (US); Ebrahim Aljohani, Somerville, MA (US)

(73) Assignee: SiPhox, Inc., Burlington, MA (US)

(21) Appl. No.: 17/859,877

(22) Filed: Jul. 7, 2022

Related U.S. Application Data
(60) Provisional application No. 63/219,018, filed on Jul. 7, 2021.

(51) Int. Cl. G01N 33/543; G02B 6/293; G02B 6/42; G01N 21/41

(52) U.S. Cl. CPC G01N 33/54373; G02B 6/29338; G02B 6/4215; G01N 21/41; G01N 2469/10; G01N 2333/165

(57) ABSTRACT

Aspects relate to devices, systems, and methods for non-invasive testing. The device may include a cartridge that analyzes a nasopharyngeal swabbing sample. The device may also include first and second waveguides, where each waveguide is configured to propagate an electromagnetic (EM) wave.

### CROSS-REFERENCE TO RELATED APPLICATIONS

This application claims the benefit of priority of U.S. Provisional Patent Application Ser. No. 63/219,018, filed on Jul. 7, 2021, and titled “IMMUNOASSAY COMBINATIONS FOR RESPIRATORY DISEASES,” which is incorporated by reference herein in its entirety.

### FIELD OF THE INVENTION

The present disclosure generally relates to systems and methods for performing chemical and biochemical assays.

### BACKGROUND

Antibody titer tests are an invasive procedure, usually done in blood serum, that may be uncomfortable for patients and may result in contamination.

### SUMMARY OF THE DISCLOSURE

In an aspect, a non-invasive testing device is provided. The device including an input configured to receive a sample; at least a first waveguide in fluidic communication with the input and configured to: propagate a first electromagnetic (EM) wave; and vary in a first optical property as a function of the sample; at least a second waveguide in fluidic communication with the input and configured to: propagate a second EM wave; and vary in a second optical property as a function of the sample; and at least a sensor in communication with the at least a first waveguide and the at least a second waveguide and configured to detect, using the first EM wave and the second EM wave, a first constituent of the sample and a second constituent of the sample, as a function of a first variance in the first optical property and a second variance in the second optical property.

In another aspect, a method of biological detection is provided. The method includes A method of detection, the method includes receiving, by an input of a housing, a sample; propagating, by at least a first waveguide in fluidic communication with the input, a first electromagnetic (EM) wave; varying, by the at least a first waveguide, in a first optical property as a function of the sample; propagating, by at least a second waveguide in fluidic communication with the input, a second electromagnetic (EM) wave; varying, by the at least a second waveguide, in a second optical property as a function of the sample; detecting, by at least a sensor in communication with the at least a first waveguide and the at least a second waveguide, using the first EM wave and the second EM wave, a first constituent of the sample and a second constituent of the sample, as a function of a first variance in the first optical property and a second variance in the second optical property.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a schematic diagram illustrating an exemplary embodiment of a testing device detection;

FIGS. 2A and 2B are schematic diagrams illustrating an exemplary embodiment of the device;

FIG. 3 is a schematic diagram illustrating an exemplary embodiment of a microring resonator sensor of the testing device

FIGS. 4A and 4B are graphs illustrating real-time binding data;

FIGS. 5A and 5B are graphs illustrating performance characteristics;

FIGS. 6A and 6B are graphs illustrating anti-protein RBD antibody detection in nasal swabs;

FIGS. 7A-7F are various graphs illustrating affinity curves and signatures for variants;

FIGS. 8A and 8B are graphs illustrating controls and sample sufficiency tests;

FIG. 9 is a flow diagram illustrating an exemplary method of antibody detection;

FIG. 10 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

Aspects of the present disclosure include a non-invasive antibody testing device that may utilize nasopharyngeal swabs and surface functionalization. The device may be chip functionalized with S protein, receptor-binding domain (RBD), or both and offers increased analytical sensitivity. Data supporting the device, systems, and methods described herein are presented and range from real-time binding data, limit-of-detection, antibody detection and comparison with prior methods, affinity and signatures of variants, and controls and sample sufficiency tests.

As used in this disclosure, a “detectable marker” is a substance that is detectable using any sensing methodology, such as and without limitation, electrical sensing, magnetic sensing, optical sensing, chemical sensing, and the like. A contrast agent may be referred to interchangeably as a label. In some cases, detectable marker may include at least one of metal nanoparticles, such as and without limitation, gold. Alternatively or additionally, detectable marker may include a fluorophore and/or a chemical dye. In some cases, detectable marker may include an optically active and/or conductive or magnetic component, which may be detectable using electronical and/or magnetic circuit elements. In one or more embodiments, detectable marker may be utilized for and/or may facilitate detection of antibody binding.

As used in this disclosure, “immobilized” refers to an attribute of no substantially relative movement between two relata. For example, and without limitation, a captured antigen, may be immobilized on a surface, such as a surface of a waveguide. In some cases, surface may be functionalized. For example, surface may be coated or otherwise treated in order to facilitate bonding, such as without limitation covalent bonding. In some exemplary embodiments, surface may be functionalized with streptavidin and/or avidin and at least an immobilizing element may include biotin, thereby facilitating immobilization. For instance, Avidin and other biotin-binding proteins, including Streptavidin and NeutrAvidin protein, have an ability to bind up to biotin molecules, thereby facilitating immobilization. The Avidin-biotin complex is a strong non-covalent interaction (K_{d}=10⁻¹⁵M) between a protein and ligand. Bond formation between biotin and Avidin can be very rapid, and once formed, may be unaffected by extremes of pH, temperature, organic solvents, and other denaturing agents. These features of biotin and Avidin—features that are shared by Streptavidin and NeutrAvidin Protein—are useful for immobilization.

As used in this disclosure, a “waveguide” is an element configured for propagation of electromagnetic waves. In some cases, a waveguide may be configured to propagate an electromagnetic (EM) wave by any of total internal reflection, attenuated total internal reflection, and/or frustrated internal reflection. In some cases, a waveguide may be configured to propagate an EM wave through reflection, transmission, and/or scattering. In some cases, a waveguide may be configured to propagate electromagnetic radiation (EMR) through surface plasmons such as, for example and without limitation, through surface plasmon resonance. Surface plasmon resonance (SPR) may include resonant oscillation of conduction electrons, for instance, and without limitation, at an interface between negative and positive permittivity material stimulated by incident light. SPR may alternatively or additionally be used to measure adsorption of material onto planar metal, such as, and without limitation, gold or silver, surfaces or onto a surface of metal nanoparticles, for instance, and without limitation, if metal nanoparticles are used as detectable marker, as described above.

As used in this disclosure, an “evanescent wave” (also referred to in this disclosure as an “evanescent field”) may result from EM wave propagation within waveguide. An evanescent wave may exhibit a rapidly decaying (or vanishing) field amplitude in a certain spatial direction, for example, orthogonal to surface of waveguides. In some cases, an evanescent wave may not contribute to energy transport in a spatial direction such as a direction in which evanescent wave exhibits a rapidly decaying or vanishing field amplitude, although in some cases a Poynting vector (averaged over one oscillation cycle) may have non-zero components in other directions. Evanescent wave may be used to detect binding, such as and without limitation, antigen and/or antibody binding. In some cases, a light signal detected by sensor may indicate presence and/or absence of a particular antigen. For example, and without limitation, an attenuated light signal may indicate that antigen having a highly absorbent detectable marker is proximal to s surface of a waveguide, as the attenuated light signal may result from evanescent wave coupling, for instance via absorption, into detectable marker. As an evanescent wave “vanishes” along a certain direction, its field amplitude, and therefore ability to be used for sensing, may diminish drastically as distance away from surface increases. For example, depending upon parameters, such as index of refraction, light wavelength, light coupling angle, to name a few, an evanescent wave may practically propagate less than 100 μm from surface, less than 10 μm from surface, or even less than 1 μm from service.

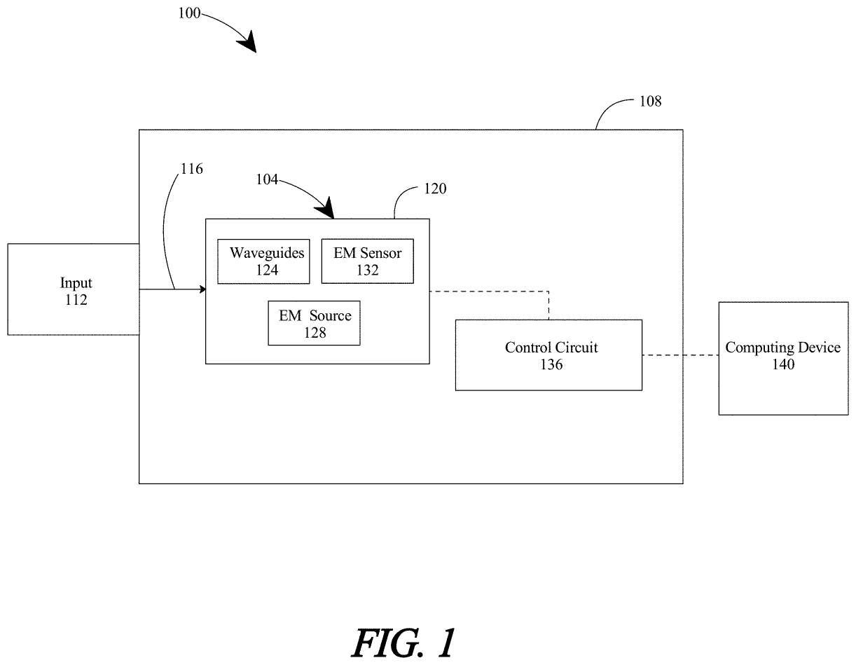

Now referring to FIG. 1 an exemplary embodiment of non-invasive testing device 100 is shown. In one or more embodiments, device 100 may include an immunoassay (IA) device. Device 100 may include a disposable cartridge, where each disposable cartridge contains a silicon chip with microring resonator sensors, as discussed further in this disclosure. Cartridge may be used in conjunction with an anterior nasal swab and extraction tube user-flow to provide high analytical sensitivity for SARSCoV-2 N protein as well as quantitative anti-RBD antibody data, Flu A, Flu B, and RSV infection status. Nasal swab eluent antibody levels as measured by device 100 correlate strongly with serum antibody levels, which may eliminate the need for an invasive fingerpick or blood draw to accurately track antibody levels. Device 100 may capture the binding affinity of S protein (if present) against four distinct captures. Subsequently, S protein affinity data is streamed to the cloud alongside the other test results and analyzed to form a unique signature, as discussed further below. Comparing each sample's S protein affinity signature to calibration data for known variants of SARS-CoV-2 enables robust first line of defense variant biosurveillance in at-home and point-of-care settings for a fraction of the cost of DNA sequencing and with a, for example and without limitation, expedited turnaround time (e.g., 15-minute). Additionally, the device 100 may be in communication with other devices via, for example, a cloud server, which may flag samples with high N protein content (i.e. high viral load) and high anti-S protein RBD IgGs as possible instances of immune escape. Biosurveillance data may thus be made readily available to public health authorities via a HIPAA compliant Web Service backend API. Instructions for testing device operation, as well as test results, may be provided to a user via a web or smartphone application. Positive, negative, and human sample sufficiency controls may be included in the assay alongside multiple software and hardware features for user-error risk mitigation.

The presence of immunoglobulins against viral antigens is a sign of adaptive immune response and protection from re-infection from the same virus. Studies show that adaptive immune response start forming quickly with production of IgM and IgA antibodies which can be detected in the blood. Antibodies can be detected in other places, such as the nose (e.g., nasopharyngeal swab) or mouth (e.g., saliva sample). Analysis of antibody content in these bodily fluids revealed that nasal swabs have the second highest concentration of IGs outside of blood. Thus, devices and systems utilizing nasopharyngeal swab-based samples are promising for safe, minimally invasive antibody testing. This includes, but is not limited, to quantifying immune protection against COVID-19 based on IgG, IgM and IgA levels in nasopharyngeal swab samples.

One aspect of the present discusses various devices, systems, and methods for performing chemical and biochemical assays on a chip-based device, such as device 100, related to antibody testing. These detection systems and schemes outlined may be implemented individually or in combination (i.e. multiplexed) and may comprise one or more integrated photonics chips (sub-chips) where light is transferred between them using a single or plurality of light transfer methods to facilitate detection.

Still referring to FIG. 1, device 100 may include a biosensor 104 that is configured to detect a target biological element, such as an antigen or an antibody, of a sample. In one or more embodiments, biosensor 104 may be at least partially contained within a housing 108 (e.g., cartridge) of device 100. A “sample” may include a biological sample of an organism, such as a human. Sample may include a biological extraction and/or specimen from the organism, such as saliva, mucus, cells, and the like, that can be extracted using various means, such as a mouth swab collecting saliva or a nasal swab collecting cells from the nasopharynx. In various embodiments, sample may be mixed into a solution prior to being inputted into device 100. In one or more embodiments, sample may be deposited into device 100 using an input 112 of device 100. In some embodiments, input 112 may include a sample reservoir. For example, and without limitation, a solution containing sample may be inputted into device 100 via a sample reservoir. Thus, reservoir may be configured to contain a fluid. In some embodiments, input be in fluidic communication with one or more channels that allow fluid, such as a solution with sample, to flow to and/or through biosensor 104, allowing the solution to come into contact with waveguides, as discussed further in this disclosure. Waveguides of device 100 may disposed in and/or proximate a region or channel that is in fluidic communication with input 112 so that sample may be received by input and transferred to biosensor, which includes waveguides. For example, and without limitation, sample solution may be deposited into sample reservoir of device 100, where once inputted into device 100, sample may traverse through one or more channels 116 of device 100, which are in fluidic communication with input and biosensor, and into and/or through biosensor 104.

Still referring to FIG. 1, system may include biosensor 104. Biosensor 104 may be configured to facilitate antigen detection of sample. Biosensor 104 may include one or more waveguides 124, an electromagnetic (EM) source 128, an electromagnetic (EM) sensor 132, and/or other components. One or more components of biosensor 104 (e.g., waveguides 124) may be mounted on a substrate, which may be at least partially contained within containment 120 of biosensor. Containment 120 may include a housing that at least partially encloses components of biosensor 104. In some embodiments, channel 116 may traverse through containment 120 to position sample near waveguides 124 for analysis. In other embodiments, channel 116 may output sample into containment 120 of biosensor 104 so that sample may directly contact and/or surround components of biosensor, such as, for example, waveguides 124.

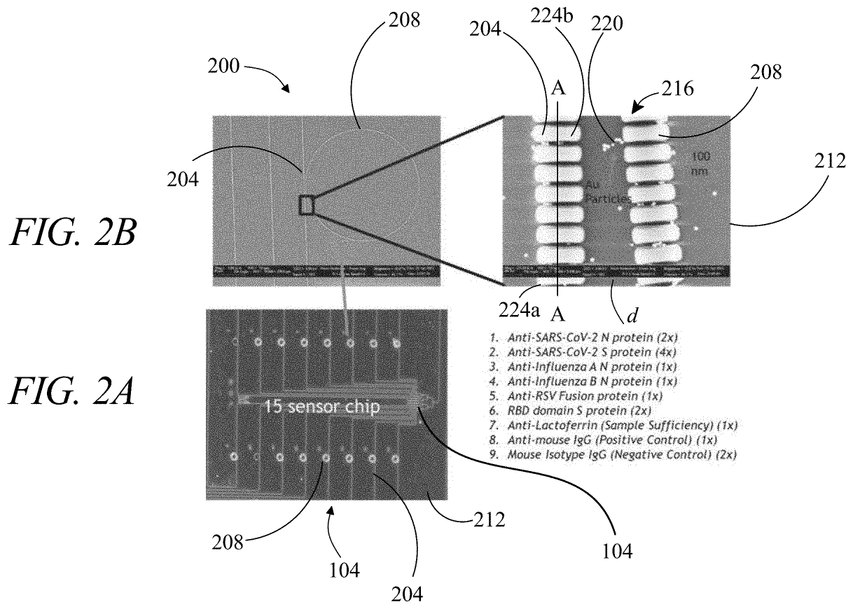

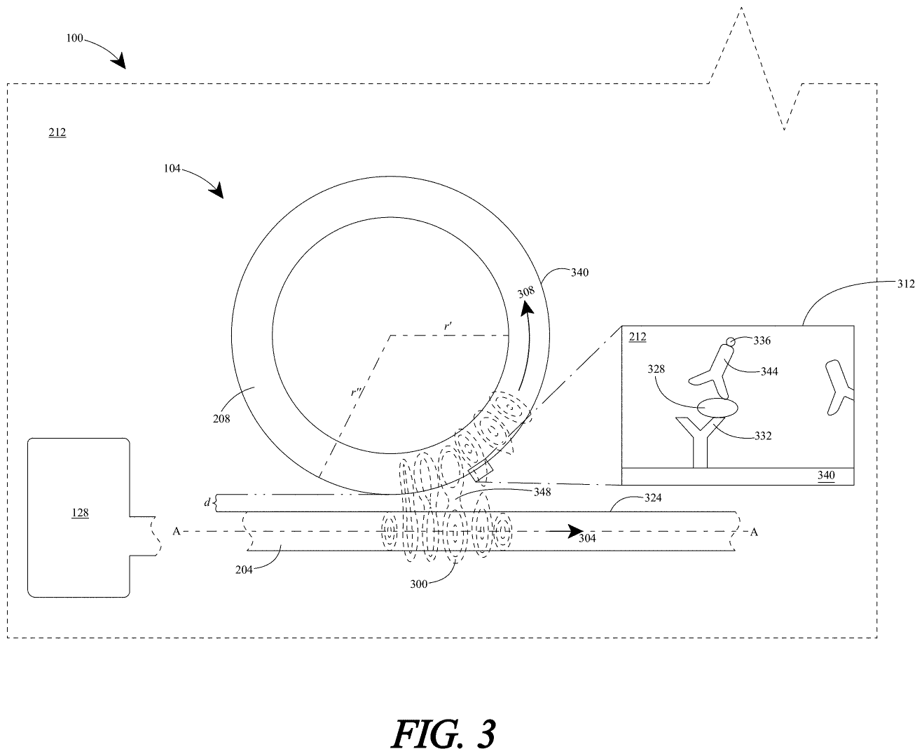

Still referring to FIG. 1, biosensor 104 may include one or more waveguides 124, which are configured to reflect and/or refract electromagnetic (EM) waves from electromagnetic source 128. EM source 128 may project EM waves into waveguides 124, as discussed further below in this disclosure. Waveguides 124 may each include a surface that, in some nonlimiting embodiments and without limitation, one or more antibodies or antigens may be immobilized upon. In some embodiments, waveguides 124 may each include an optical waveguide configured to propagate EM waves, such as and without limitation, light by way of total internal reflection. Accordingly, in some cases, waveguides 124 may each have an index of refraction that is substantially greater than a medium surrounding waveguides 124. For example, and without limitation, in some exemplary cases, waveguides 124 may include sapphire and have an index of refraction which is greater than 2, and medium surrounding waveguides 124 may include water and have an index of refraction of about 1.4. In one or more embodiments, waveguides 124 may include a plurality of waveguides. For example, and without limitation, waveguides 124 may include a pair of waveguides, as shown in FIG. 3. In another example, and without limitation, waveguides 124 may include thirty waveguides, as shown in FIG. 2A.

Still referring to FIG. 1, biosensor 104 may include electromagnetic (EM) source 128, which is configured to output an EM wave that interacts with waveguides 124, as discussed further below in FIG. 3. Electromagnetic waves from EM source 128 may be emitted into and/or projected onto waveguides 124, as discussed further in this disclosure below. In various embodiments, EM source 128 may include a light source. Light source may be configured to emit a light, which may be emitted into and/or at waveguide. As used in this disclosure, a “light source” is any device configured to emit a light. Light source may include a coherent light source and/or an incoherent light source. Nonlimiting exemplary light sources include lasers, light emitting diodes (LEDs), organic LEDs (OLEDS), light emitting capacitors, incandescent lamps, fluorescent lamps, and the like. system 100 includes first light source 108. In various embodiments, device 100 may include a plurality of light sources. In some cases, light source may include a coherent light source, which is configured to emit coherent light, for example, a laser. In some cases, light source may include a non-coherent light source configured to emit non-coherent light, for example a light emitting diode (LED). In some cases, light source may emit a light having substantially one wavelength. In some cases, light source may emit a light having a wavelength range. Light may have a wavelength in an ultraviolet range, a visible range, a near-infrared range, a mid-infrared range, and/or a far-infrared range. For example, in some cases light may have a wavelength within a range from about 100 nm to about 20 micronmeters. In some cases, light may have a wavelength within a range of about 400 nm to about 2,500 nm. Light sources may include, one or more diode lasers, which may be fabricated, without limitation, as an element of an integrated circuit; diode lasers may include, without limitation, a Fabry Perot cavity laser, which may have multiple modes permitting outputting light of multiple wavelengths, a quantum dot and/or quantum well-based Fabry Perot cavity laser, an external cavity laser, a mode-locked laser such as a gain-absorber system, configured to output light of multiple wavelengths, a distributed feedback (DFB) laser, a distributed Bragg reflector (DBR) laser, an optical frequency comb, and/or a vertical cavity surface emitting laser. Light source may additionally or alternatively include a light-emitting diode (LED), an organic LED (OLED) and/or any other light emitter. In some cases, first optical output and second optical output may be combined along a shared optical path. Light 136 may be coupled into waveguide and propagate within waveguide, for example and without limitation using total internal reflection. In some cases, light may exit waveguide and be detected by a sensor, such as EM sensor 132, upon exiting waveguide. Waveguide may include any structure that may guide waves, such as electromagnetic waves or sound waves, by restricting at least a direction of propagation of the waves. Waves in open space may propagate in multiple directions, for instance in a spherical distribution from a point source. A waveguide may confine a wave to propagate in a restricted sent of directions, such as propagation in one dimension, one direction, or the like, so that the wave does not lose power, for instance to the inverse-square law, while propagating, and/or so that the wave is directed to a desired destination such as a sensor, light detector, or the like. In an embodiment, a waveguide may exploit total reflection at walls, confining waves to the interior of a waveguide. For example, and without limitation, waveguide may include a hollow conductive metal pipe used to carry high frequency radio waves, such as microwaves. Waveguide may include optical waveguides that, when used at optical frequencies, are dielectric waveguides whereby a structure with a dielectric material with high permittivity and thus a high index of refraction may be surrounded by a material with a material with lower permittivity. Such a waveguide may include an optical fiber, such as used in fiberoptic devices or conduits. Optical fiber may include a flexible transparent fiber made from silica or plastic that includes a core surrounded by a transparent cladding material with a lower index of refraction. Light may be kept in the core of the optical fiber by the phenomenon of total internal reflection which may cause the fiber to act as a waveguide. Fibers may include both single-mode and multi-mode fibers. Acting as a waveguide, fibers may support one or more fined transverse modes by which light can propagate along the fiber. Waveguides may be made from materials such as silica, fluorozirconate, fluoroaluminate, chalcogenide glass, sapphire, fluoride, and/or plastic.

Still referring to FIG. 1, device biosensor 104 of device 100 may include one or more EM sensors. In various embodiments, EM sensor 132 may be placed along one or more of waveguides 124. In other embodiments, EM sensor 132 may be positioned proximate to one or more of waveguides 124. In various embodiments, EM sensor 132 may include any photon detector. In some cases, an output of optical waveguides 124, such as an output of a linear waveguide, may be coupled to a photodetector. EM sensor 132 may include any optical or EM sensor, including without limitation a photosensor, a photodetector, a thermopile, a pyrolytic sensor, a photodiode, avalanche photodiode (APD), single photon avalanche photodiode (SPAD), and the like.

Still referring to FIG. 1, as used in this disclosure, a “sensor” may include any device that is configured to detect a phenomenon, for example a phenomenon associated with light interacting with at least a detectable marker, as discussed further in this disclosure. A sensor may include any type of sensors, including, and without limitation, sensors configured to detect electrical phenomenon, chemical phenomenon, and/or optical phenomenon. General sensing techniques may include, but are not limited to, using a doped optical waveguide or electrodes near a waveguide to sense an optical change or resistance change, respectively, before or after binding. In some examples, optical changes may be detected using surface plasmon resonances, Mach-Zehnder interferometers, spiral waveguides, Bragg gratings, and/or photonic crystals or magnetic dielectric mirrors. In some embodiments, sensors may be collocated with surfaces of waveguides and/or of a substrate that waveguides are integrated into and/or mounted thereto; said another way, surfaces of one or more waveguides 124 may include the sensor 132 or a detection path between the sensor and the at least detectable marker, antigen, or antibody. In one or more embodiments, sensor, such as EM sensor 132 may be in communication with waveguides 124 and/or one or more elements of sample. As used in this disclosure, “communication” is used to refer to a causal or sensed relationship between two relata. Communication may include communication of information and/or data. Communication may also include fluidic communication, where a fluid may be moved between two or more components of device 100. In a nonlimiting example, a photosensor may be said to be in communication with a detectable marker that is reflecting, fluorescing, or otherwise transmitting a light, which the photosensor is detecting, as discussed further below in this disclosure.

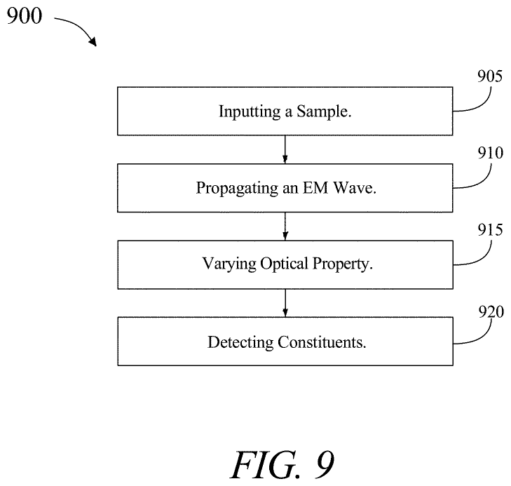

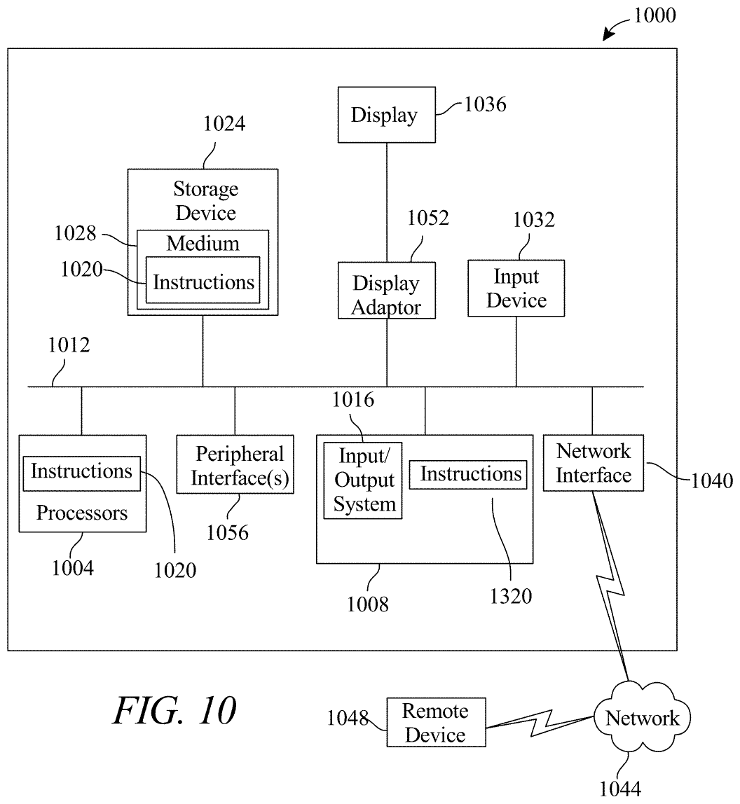

Still referring to FIG. 1, device 100 may include a control circuit. In one or more embodiments, EM sensor 132 may be communicatively connected to a control circuit 136 and/or a remote device, such as a user device. Control circuit 136 may include an analog circuit, a computing device, a processor (e.g., processor 1004 shown in FIG. 10), a microprocessor, and the like. Circuit 136 may take as input a signal from sensor 132 and process the signal. In some cases, one or more optical elements and/or optical devices may be used to couple EM radiation or light into and/or out of waveguide. For example, and without limitation, coupling lenses having a numerical aperture selected based upon acceptable entrance angle and/or cross-sectional area of waveguide may be used to couple substantially collimated light into waveguide and/or substantially collimate light after exiting waveguide. Circuit may include analog and/or digital circuit elements. Exemplary nonlimiting analog elements include operational amplifiers, comparators, amplification circuits, and the like. In some cases, circuit may include an analog circuit interfaced with a digital circuit, for example and without limitation, by way of an analog to digital (A/D) converter. Alternatively or additionally, an analog circuit may be interfaced with a digital circuit by way of a resistive divider, such as without limitation a Wheatstone bridge. Alternatively or additionally, analog circuit may be interfaced with a digital circuit by way of at least a control terminal of transistors (or other digital elements), which are configured to trip (or otherwise digitally indicate) a certain voltage threshold to configured to be indicative of a change in digital state. In some cases, circuit may include a digital circuit. Digital circuit could be any combinatorial or sequential circuit including logic gates, registers, and the like. Digital circuit may include a microprocessor, microcontroller, or the like. Digital circuit may include connection to a memory. In some embodiments, digital circuit may include or interface with at least a computing device. Computing device may include any computing device described in this disclosure, for example with reference to FIG. 9. In some cases, device 100 may be configured with aid of a computing device to perform any methods, steps, and/or processes described in this disclosure automatically. In one or more embodiments, control circuit may include or be communicatively connected to a computing device, such as a remote computing device. A remote computing device may include a user device, such as a laptop computer, desktop computer, tablet, or other device configured to implement a graphic user interface.

With continued reference to FIG. 1, control circuit may be communicatively connected to or a component of a computing device. Computing device may include any computing device as described in this disclosure, including without limitation a microcontroller, microprocessor, control circuit, digital signal processor (DSP), and/or system on a chip (SoC) as described in this disclosure. For example, and without limitation, computing device may include computing system 1000, shown in FIG. 10. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device may include but is not limited to, for example, a computing device or cluster of computing devices in a first location and a second computing device or cluster of computing devices in a second location. Computing device may include one or more computing devices dedicated to data storage, security, distribution of traffic for load balancing, and the like. Computing device may distribute one or more computing tasks as described below across a plurality of computing devices of computing device, which may operate in parallel, in series, redundantly, or in any other manner used for distribution of tasks or memory between computing devices. Computing device may be implemented using a “shared nothing” architecture in which data is cached at the worker, in an embodiment, this may enable scalability of device 100 and/or computing device.

With continued reference to FIG. 1, computing device may be designed and/or configured to perform any method, method step, or sequence of method steps in any embodiment described in this disclosure, in any order and with any degree of repetition. For instance, computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

FIGS. 2A and 2B show an exemplary embodiment of a biosensor 104 of device 100. In one or more embodiments, device 100 may include a disposable cartridge that has a silicon chip, such as biosensor 104, that leverages the datacom supply chain. FIG. 2A shows an exemplary biosensor chip 104 that includes a 15-sensor chip. Biosensor 104 may include one or more microring resonator sensors 200 (e.g., 15 microring resonator sensors) Each microring resonator sensor 200 may include one or more waveguides, such as waveguides 124 (shown in FIG. 1). In one or more embodiments, each microring resonator sensor may include a plurality of waveguides, such as a first waveguide 204 and second waveguide 208, used for detection purposes. In various embodiments, microring resonator sensors may be mounted to a cladding or substrate, such as substrate 212.

FIG. 2B shows an illustration of a biosensor 104 of device 100 (after 20 pm N protein nasal swab sample exposure), where first waveguide 204 is optically coupled with second waveguide 208. For the purposes of this disclosure, “optically coupled” or “optical coupling” is a method of interconnecting two or more devices to transfer an optical signal using EM waves, such as light waves. Inset 216 of FIG. 2B shows a close-up of a coupling region of first waveguide 204 and second waveguide 208, where first waveguide 204 and second waveguide 208 are positioned adjacent to and a distance d (e.g., distance gap) from each other. In one or more embodiments, device 100 includes first waveguide 204, which is configured to allow for an electromagnetic (EM) wave (e.g., light) to traverse therethrough. For example, and without limitation, a first EM wave 348 may propagate through first waveguide 204. First waveguide 204 may include a linear waveguide (e.g., a substantially straight waveguide or a channel waveguide) that extends along a longitudinal axis A of a body (e.g., length) of first waveguide 204 and includes a first end 224a (e.g., an input end) and a second end 224b (e.g., an output end). In one or more embodiments, a light source may be positioned at first end 624a so that light (such as light 300 of FIG. 3) may traverse through first waveguide 204 from first end 224a to second end 224b (as indicated by directional arrow 304 of FIG. 3). In various embodiments, a sensor 132, such as an optical sensor or photodiode, may be positioned at second end 224b of first waveguide 204. Through an evanescent field 348 and coupling, a second EM wave 352 may propagate through second waveguide 208 (e.g., propagating about a central axis of second waveguide 208 as indicated by direction arrow 308), where second EM wave 352 may have differing optical properties from first EM wave 348. Optical properties may include optical properties of matter, such as refraction index, dispersion, fluorescence, perceived color, photosensitivity, and the like, that may be detected by a sensor, such as EM sensor 132. In other embodiments, light source 128 may be positioned proximate to first waveguide 204 and second waveguide 208. In one or more embodiments, first waveguide 204 and/or second waveguide 208 may be grated (e.g., a grating coupler), as shown in inset 216 of FIG. 2B, which may facilitate waveguide-mode resonance.

Now referring to FIG. 3, an exemplary embodiment of microring resonant sensor 200 is shown. In one or more embodiments, device 100 and biosensor 104 may include second waveguide 208, which is configured to allow an EM wave, such as light, to propagate therethrough, where the EM wave may be received by second waveguide 208 through an evanescent field 348 from first waveguide 204. Second waveguide 208 may include a circular or toroidal waveguide having an inner radius of r′ and an outer radius of r″. For example, and without limitation, second waveguide 208 may include a ring resonator (also referred to in this disclosure as a “microring”, “microring resonator”, or “microtoroid”). Second waveguide 208 may be positioned adjacent or optically coupled to first waveguide 204. In one or more exemplary embodiments, light 300 may propagate through first waveguide 204 so that light is inputted from light source at a first end 224a of first waveguide 204 and is then received and/or outputted by second end 224b of first waveguide 204. Optical coupling occurs within second waveguide 208 as the input mode propagates past second waveguide 208 so that an EM wave may propagate through second waveguide 608 (as shown by directional arrow 308). Thus, some of the light wave reflected within first waveguide 204 may be coupled into second waveguide 208 through an evanescent field phenomenon, where light extends outside first waveguide in, for example, an exponentially decreasing radial profile. In some embodiments, a light sensor may be positioned at the second end 224b of first waveguide 204. First waveguide 204 may be configured to propagate a first electromagnetic (EM) wave and vary in a first optical property as a function of the sample. Second waveguide 208 is configured to propagate a second EM wave and vary in a second optical property as a function of the sample, where the optical property may include a wavelength, frequency, intensity, and the like.

Still referring to FIG. 3, in one or more embodiments, second waveguide 208 may be composed of various materials, such as, for example, silicon or glass. In various embodiments, each device 100 (e.g., 5×5 mm, contained within a disposable cartridge, such as housing 108) may include multiple ring resonator sensors 200, as shown in FIG. 2A. Biosensor 104 may be composed of, for example, silicon waveguides, where sensors, such as an EM sensor, detects refractive index changes near surface 324 of first waveguide 204 or a surface 340 of second waveguide 208. In one or more embodiments, an array of captured antibodies 328 (shown in inset 312) may be microprinted (e.g., ˜1 nanoliter/sensor) onto sensor 132 to make them specific to a target antigen or antibody (shown in list of FIG. 2A). As understood by one of ordinary skill in the art, though described as a one-channel structure, ring resonator sensor 104 of device 100 may also use a two-channel structure, which may include two opposing linear waveguides positioned on either side of one or more ring resonators. In some embodiments, ring resonator may be a separate component from linear waveguide. In other embodiments, ring resonator may be integrated into linear waveguide by ring resonator being constructed by folding and/or looping of linear waveguide. Using a ring resonator may allow for more compact packaging, more accurate results, more sensitive detection, and the like.

In one embodiment, SARS-CoV-2 antigen detection in body fluids (e.g., blood, serum, plasma, saliva, nasopharyngeal swab, and the like) may be performed. For example, and without limitation, an antigen or viral protein (e.g., N protein, S protein, and the like) may be detected using a “sandwich” immunoassay, where a “sandwich” immunoassay includes a photonic waveguide (e.g., ring resonator) coated with a capture antibody capable of selectively binding to the antigen desired for detection. In some cases, detection of the antibody may be enhanced by conjugating it with a detectable marker, such as a contrast agent (e.g., gold nanoparticles, Horseradish peroxidase, magnetic nanoparticles, and the like).

Additionally or alternatively, antibodies against certain antigens (e.g., SARSCoV-2 N protein, S protein, S protein RBD domain, and the like) may be detected by first attaching the antigens 332, e.g., capture antigens, to photonic waveguide (e.g., microring resonator) surface 340. In some examples, anti-human secondary antibodies, e.g., detection antibodies 344, may be used to amplify the signal or to attain specificity to a certain antibody type (e.g., IgG, IgA, IgM, and the like). In some cases, detection antibodies 344 may be conjugated with a detectable marker 336, such as a contract agent (e.g., gold nanoparticles, Horseradish peroxidase, magnetic nanoparticle, and the like) to improve the signal detected by sensor 132, as previously discussed in this disclosure. In one or more embodiments, EM sensor 132 may be in communication with the at least first waveguide and the at least second waveguide and configured to detect, using first EM wave 348 and second EM wave 352, a first constituent of the sample and a second constituent of the sample, as a function of a first variance in the first optical property and a second variance in the second optical property, as discussed further below. In one or more embodiments, device 100 may include a multiplexor, which may be in communication with the at least a first waveguide 204, the at least a second waveguide 208, and the sensor 132, wherein the multiplexor is configured to multiplex the first EM wave 348 and the second EM wave 352.

In one or more nonlimiting embodiments, device 100 may include a disposable cartridge (e.g., housing 108) configured to contain a dried reagent pad (e.g., substrate 212) with detection-antibody conjugated to colloidal gold, such as colloidal gold 220 of FIG. 2B. Antigen binding to a surface (e.g., surface of dried reagent pad, first waveguide, second waveguide, interior surface of housing, a surface of a channel extending through biosensor, and the like) may create a small signal, which is amplified dramatically when colloidal gold particles bind to form a sandwich (as shown inset 216 of FIG. 2B and inset 312 of FIG. 3). In some cases, a few gold particles are sufficient to produce a detectable signal in complex matrix. Due to open fluidic channels in device 100, aggregation of gold particles will not cause clogging (as can happen in lateral flow strips when a high concentration of antigen is present), making highly multiplexed systems feasible. The similarity of the assay configuration to lateral flow assays enables an inexpensive, simplified cartridge with completely passive flow driven by a wicking pad, while delivering real-time, multiplexed data (e.g., the sensors are interrogated every second). A cartridge with passive flow may be a cartridge disclosed in Patent No. XX, filed XX and entitled “ [symbol] ” the entirety of which is incorporated by reference.

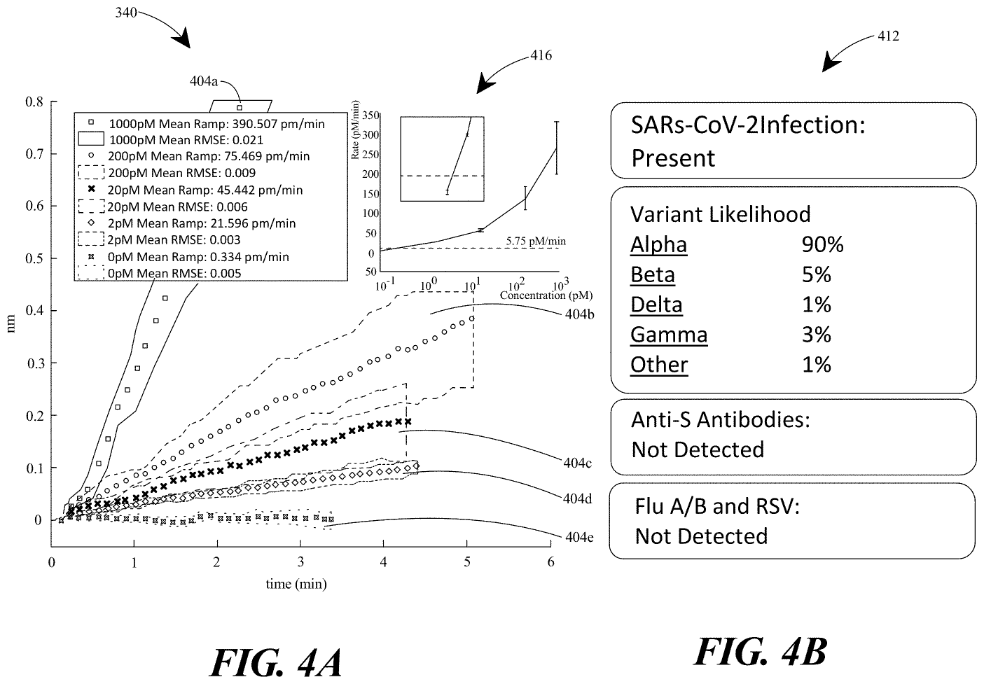

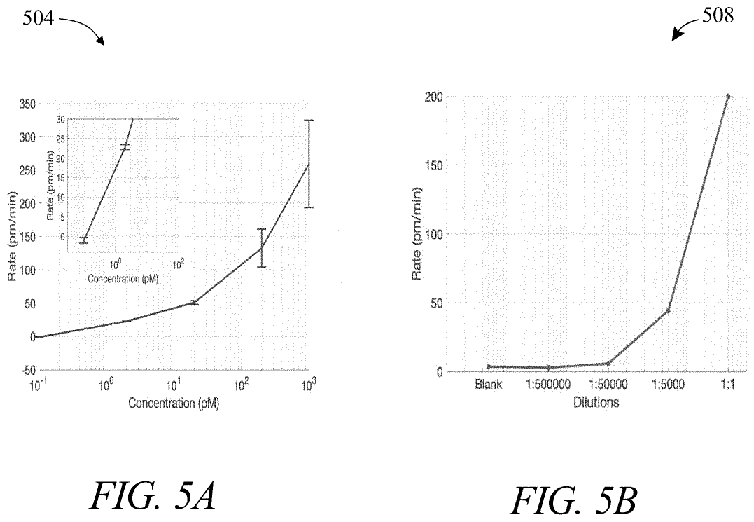

Now referring to FIG. 4A, a graph 402 showing real-time data from a binding of pooled nasal swab eluent samples spiked with gamma irradiated virus at varying concentrations is shown. More specifically, graph 402 shows binding data for SARS-CoV-2 N protein, such as binding rates for each biosensor. Binding rates 404a-e may be either compared to a cut-off value to give a positive or negative result or compared to a standard curve to give quantitative results, such as shown in table 412 of FIG. 4B. Inset graph 416 shows the corresponding concentration curve (also shown in FIG. 5A).

Now referring to FIG. 4B, results from device 100 may be presented to a user via a graphical user interface (GUI) 412. For example, and without limitation, results from a test may be readily available on a personalized app accessed using, for example, a remote device, such as a computing device (shown in FIG. 10), smartphone, laptop computer, desktop computer, tablet, and the like. In some embodiments, result data may be made accessible via a web application accessible of public health authorities, as shown in exemplary result display shown in FIG. 4B. In some cases, test results may be presented to a user as a qualitative value, for example, anti-spike IgG levels may be shown as HIGH, MEDIUM, or LOW. In other embodiments, results may be presented to a user as quantitative and/or numerical values, such as percentages, quantities, and the like. In one or more embodiments, variant biosurveillance may be accomplished via multiple sensors, where each sensor is functionalized with different S protein binders and real-time binding kinetics may be post-processed by, for example, a remote computing device, to form a signature based on the structure of the S protein in the sample. Uniquely, this approach doesn't require any foreknowledge about the variant, providing a critical early warning system and sample/patient triage tool. This approach may additionally flag samples with high levels of anti-S IgGs and N protein as potential instances of immune escape.

Now referring to FIGS. 5A and 5B, preliminary studies were performed to determine a limit of detection (LOD). The studies were conducted using an approach of testing from an upper limit of 1 nM down to a lower limit of 2 pM for SARS-CoV-2 N protein (5.23×10⁵ genome copies/ml) in a 15-minute assay format using gamma irradiated virus-spiked nasal swab samples collected from vaccinated individuals. Graph 504 of FIG. 5A shows the SARS-CoV-2-gamma irradiated virus concentration curve (given in molar equivalents of N protein). The theoretical LOD, calculated from the concentration curve, is 230 fM (6.0×10⁴ copies/ml) and testing down to 500 fM using recombinant N protein has been conducted. Device 100 is also proven to detect the presence of anti-S protein antibodies, allowing assessment of vaccination and past infection status. In a group of eight samples, vaccinated individuals have 4 times higher signal than previously infected individuals and 8 times the signal of unvaccinated individuals. Graph 508 of FIG. 5B shows a Flu A concentration curve, given in Flue A lysate dilutions. The Flu A assay has been validated down to a 50,000:1 dilution of viral lysate.

For the study, nasal swabs were obtained from ten anonymous volunteers and each swab was immediately eluted in 500 μL of lysis buffer and pooled. Samples were immediately prepared by adding gamma-radiation inactivated SARS-CoV-2 (BEI Resources NR-52287 SARS-Related Coronavirus 2, Isolate USAWA1/2020, Gamma-Irradiated) at 1 nM, 200 pM, and 2 pM N protein (2.61×10⁸, 5.23×10⁷, and 5.23×10⁵ genomic copies/mL) and tested with an exemplary embodiment of device 100, which was functionalized with anti-SARS-CoV-2 N protein antibody. As shown in graph 504 of FIG. 5A, the theoretical LOD was derived from the standard curve to be 230 fM or 6.0×10⁴ copies/ml. Furthermore, experiments with nasal swab eluent spiked with recombinant N protein were separately performed down to 500 fM. A side-by-side comparison using spiked pooled nasal swab eluent with 2 EUA approved antigen tests (Abbott BinaxNOW and Access Bio Carestart) shows the analytical sensitivity of device 100 was at least an order of magnitude better than conventional tests.

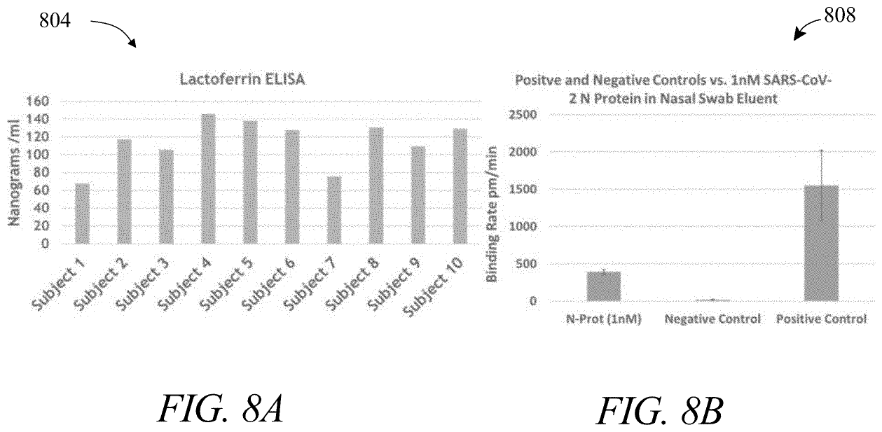

Thus, Influenza A Nucleoprotein (NP) was detected with mouse antiinfluenza NP antibody, immobilized on chip, and mouse anti-influenza A gold colloid. A standard curve, right pane of FIG. 8B, was generated by serially diluting Fapon Biotech, Inc. inactivated Influenza A lysate (Fapon Biotech, Inc. Cat #GRNINF105) in pooled nasal swab sample to make the following standard curve: stock lysate, a 1:5000 dilution in lysis buffer, a 1:50000 dilution in lysis buffer, and a 1:500000 dilution in lysis buffer.

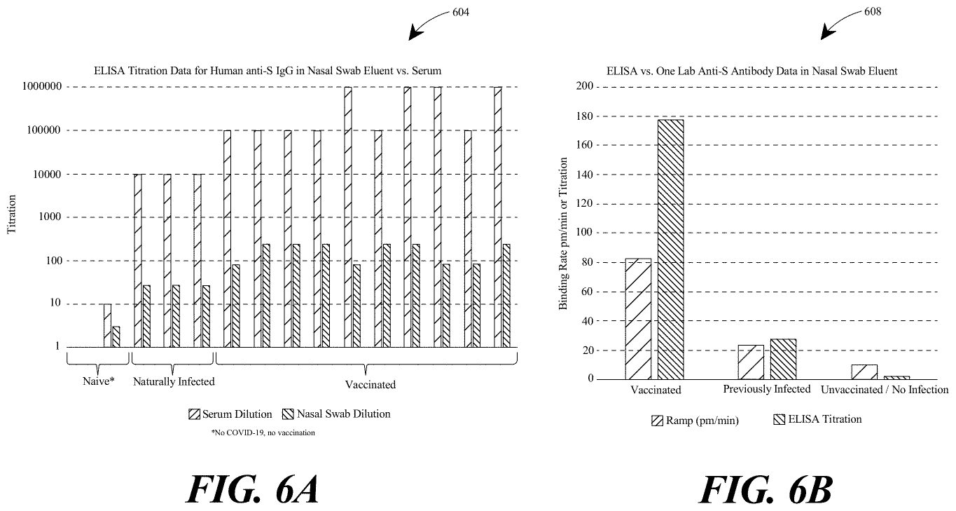

Human immunoglobulins G (IgGs) against SARS-CoV-2 S protein demonstrate the presence of an immune response to the virus either due to a natural response to infection or vaccination. To validate the approach described herein, an FDA EUA approved test (WANTAI SARS-CoV-2 Ab ELISA) was used to test 15 subjects for the presence of anti-RBD antibody in human serum. Among these 15 subjects, ten were vaccinated donors, three donors had natural immunity as a result of infection, and two were unvaccinated and previously uninfected donors. All donors donated blood and nasopharyngeal swab (NS). It was found that 13 donors (previously infected or vaccinated) had antibodies in serum and 2 (no COVID-19 history) did not. Next, a SiPhox developed enzyme-linked immunosorbent assay (ELISA) was used to compare anti-RBD antibody levels in the donor serum and NS samples. As seen in FIG. 6A, it was found that vaccinated people have anti-RBD antibody levels higher than subjects who recovered from SARS-CoV-2 and SARSCoV-2 naive donors had near-background levels.

Now referring to FIGS. 6A and 6B, graphs 604 and 608 relating to anti-protein RBD antibody detection from nasal swabs are shown. Graph 604 of FIG. 6A shows titers data for serum and nasal swab eluent samples across fifteen anonymous subjects, where the y-axis is a log scale. Graph 608 of FIG. 6B shows average values for nasal swab titers and device 600 binding rate across all subjects split into three immunity categories. The three categories including: naive (e.g., neither vaccinated nor infected with SARS-CoV-2), COVID-19 patients (e.g., had COVID-19 one month before the test or earlier, and vaccinated. The approach previously described is able to correctly quantify antibodies in all three sample types. The slightly higher background in the naive samples than what is seen in ELISA may be explained by the lack of a wash step and may be eliminated if the negative control is taken into account. Given that anti-S IgGs are typically not present in patients until 10 or more days after infection, a cloud server may flag samples with high anti-S protein IgG levels and positive N protein test results as possible immunity breakthrough infections. It is worth noting that the dynamic range of the OneLab allows the detection of anti-S protein antibodies without sample dilution, whereas the ELISA run on the same samples required dilutions up to 230 times the original concentration.

The relative binding affinity of S protein variants to the ACE2 receptor has been found to correlate with infectivity. S proteins from the Wuhan, UK, South African, Brazilian and Indian strains exhibit significantly varying binding affinities to antibodies against the RBD domain as well as to the ACE2 receptor. Using the relative binding rates of different captures, a unique signature was generated (normalized to the Wuhan variant) for each known variant and it is reasonable to assume that novel variants with sufficient mutations will exhibit unique signatures as well. In combination with the quantitative anti-S antibody assay described previously, such as device 600, variant binding signatures may offer a powerful public health monitoring tool.

As previously mentioned in this disclosure, variant S proteins may be detected using ACE2 protein attached to a photonic waveguide (e.g., microring resonator (MMR)) surface. In some examples, a detection antibody conjugated to a detection marker may be used to improve the signal. In some examples, detected viral variants may be distinguished based on their affinity to the receptor compared with the affinity to a standard anti-S protein antibody (i.e. antibody which has similar affinity to all variant proteins). In another example, viral variants may be distinguished using competitive immunoassay, in which ACE2 protein is pretreated with a competitive agent (e.g., S protein from Wuhan-Hu-1 SARS-CoV-2 isolate, angiotensin II, and the like) conjugated to a detectable marker (e.g., gold nanoparticles, Horseradish peroxidase, magnetic nanoparticles etc.) to improve the signal.

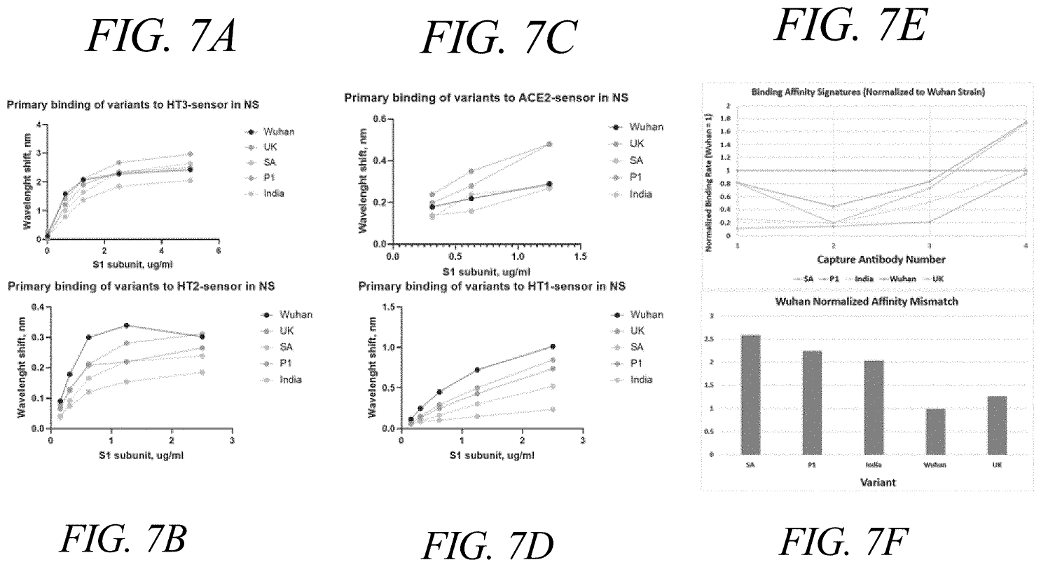

Now referring to FIGS. 7A-10F, bindings data is shown. FIGS. 7A-7D show affinity curves for five variants to three antibodies or ACE2 as captures. In an initial validation of this approach related to relative binding affinity of S protein variants to the ACE2 receptor, nasopharyngeal swabs were obtained just prior to the assay from 10 anonymous volunteers and each swab eluted in 500 μL of lysis buffer and pooled. Recombinant S protein S1 subunits from the Wuhan, UK (alpha), South Africa (beta), Brazil (gamma) and India (delta) variants were added to individual aliquots at varying concentrations (AcroBiosystems, cat #S1N-C₅₂H_{3,} S1N-C₅₂Hm, S1NC₅₂Hp, S1N-C₅₂Hr, S1N-C52Ht). The samples were then analyzed on a Gator Bio biolayer interferometer, which uses a resonant biosensor that was functionalized with the exact surface chemistry used for device 100. The samples were interrogated with biosensors coated with 4 anti-S protein antibodies and recombinant human ACE2 (SinoBiological, cat #10108-H05H). FIGS. 7E shows affinity signatures for each variant normalized to the Wuhan variant. FIG. 7F shows the difference between the Wuhan signature and other variant signatures as calculated via the Kolmogorov-Smirnov test.

In some examples, a sample sufficiency test, where enough biological sample material is present for detection, may be performed using a detection target, such as, for example, an antibody, against a common protein enriched in the body fluid (e.g., lactoferrin for nasopharyngeal swab, albumin for blood etc.) attached to a photonic waveguide, such as, for example, an MMR. For example, and without limitation, detection of common bodily fluid would indicate adequate biological sample to confirm a negative test (e.g., no antibody detected), while no detection of the common bodily fluid (e.g., lactoferrin) would indicate the sample insufficiency. Additionally or alternatively, a positive control may be performed by utilizing antibodies against the primary antibody's species (e.g., human, mouse, goat, and the like) can be used as positive control. Because these antibodies recognize only the species and do not depend on antibody target, they present a test for correct testing conditions (e.g., correct sample fluid flow through the device). Additionally or alternatively, a negative control may be performed by utilizing isotype control antibodies matching the primary antibody's species (e.g., human, mouse, goat, and the like). The isotype (e.g., IgG, IgM, IgA, and the like) may then be used to differentiate a non-specific background signal (e.g., from the primary antibody) from specific antibody signal (e.g., for target antigen), because isotype control antibodies have no relevant specificity to the target antigen.

Now referring to FIGS. 8A and 8B, control and sample sufficiency test results are shown. In a nonlimiting example of a controls validation, a positive control microring may be printed with goat anti-mouse IgG which captures gold colloid from the reagent pad. It may serve as a test for the assay, the sensitivity of the sensors, rehydration of dried reagent pad and microfluidics functionality. A mouse IgG-isotype control serves as a negative control, yielding only nonspecific background from the matrix. The response to 1 nM N protein in nasal swab eluent was compared on the mouse IgG negative control ring and the N protein detection ring. FIG. 8A, shows a graph 804 illustrating ELISA results for lactoferrin concentrations in 10 anonymized nasal swab eluent samples. As seen in FIG. 8A, assay reagents and N protein have very low binding to the negative control ring and the positive control ring has high signal. Tests that deviate significantly from acceptable ranges for the negative and positive control may be reported as invalid.

Lactoferrin is found in nasal secretions at high levels. In one nonlimiting example of a sample sufficiency validation, an initial test of the subject-to-subject reproducibility of Lactoferrin levels were performed as a sample sufficiency test. Lactoferrin levels were determined using RayBio®Human Lactoferrin kit Cat #ELH-LTF according to the kit directions. Nasal swabs were obtained just prior to the assay on ten anonymous volunteers and the samples diluted in the kit assay buffer. FIG. 8B shows triplicates of positive and negative controls alongside 1nM SARS-CoV2 N protein all in pooled nasal swab eluent. Graph 808 of FIG. 8B demonstrates that nasal swab samples yield similar lactoferrin levels with a mean of 543 ng/mL and a CV of 14.1% between subject, validating our use of lactoferrin as a control for human nasal swab content. Tests that are more than 50% below the expected minimum signal for lactoferrin may be reported as invalid.

Referring now to FIG. 9, a method 900 of biological detection is illustrated by way of a flow diagram. At step 905, method 900 may include receiving, by an input of a housing, a sample.

At method 910, method 900 may include propagating an EM wave. Method 910 may include propagating, by at least a first waveguide in fluidic communication with the input, a first electromagnetic (EM) wave. Method 910 may also include propagating, by at least a second waveguide in fluidic communication with the input, a second electromagnetic (EM) wave.

At method 915, method 900 may include varying in an optical property. Method 915 may also include varying, by the at least a first waveguide, in a first optical property as a function of the sample. Method 915 may also include varying, by the at least a second waveguide, in a second optical property as a function of the sample. The at least a second waveguide comprises a ring resonator. The at least a first waveguide is configured to provide communication between the first EM wave and a portion of the sample by propagating an evanescent wave from a surface. The first optical property includes an index of refraction of the at least a first waveguide. The first optical property includes an index of refraction of the at least a first waveguide. The at least a first waveguide is configured to provide communication between the first EM wave and a portion of the sample by propagating surface plasmons upon a surface. A first surface is coated with a capture antibody configured to selectively bind to a first antigen and vary the first optical property. The microfluid device is configured to contain a first marker, wherein the first marker is configured to selectively conjugate the first antigen and vary the first optical property.

At method 920, method 900 may include detecting, by at least a sensor in communication with the at least a first waveguide and the at least a second waveguide, using the first EM wave and the second EM wave, a first constituent of the sample and a second constituent of the sample, as a function of a first variance in the first optical property and a second variance in the second optical property. The device may further include a multiplexor in communication with the at least a first waveguide, the at least a second waveguide, and the sensor, wherein the multiplexor is configured to multiplex the first EM wave and the second EM wave. The device further includes a computing device in communication with the sensor and configured to receive, from the at least a sensor, at least a signal representing the at least a first constituent and the at least a second constituent, and communicate, with a remote device, at least a first datum representing the at least a first constituent and at least a second datum representing the at least a second constituent. The remote device is configured to process the at least a first datum and the at least a second datum and recognize a variant of the substance, as a function of processing the at least a first datum and the at least a second datum.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve embodiments according to this disclosure. Accordingly, this description is meant to be taken only by way of example, and not to otherwise limit the scope of this invention.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Processor 1004 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Memory 1008 may include various components (e.g., machine-readable media) including, but not limited to, a random-access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 1094 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A non-invasive testing device, the device comprising:
an input configured to receive a sample;
at least a first waveguide in fluidic communication with the input and configured to:
propagate a first electromagnetic (EM) wave; and
vary in a first optical property as a function of the sample;
at least a second waveguide in fluidic communication with the input and configured to:
propagate a second EM wave; and
vary in a second optical property as a function of the sample; and
at least a sensor in communication with the at least a first waveguide and the at least a second waveguide and configured to detect, using the first EM wave and the second EM wave, a first constituent of the sample and a second constituent of the sample, as a function of a first variance in the first optical property and a second variance in the second optical property.

2. The device of claim 1, further comprising a multiplexor in communication with the at least a first waveguide, the at least a second waveguide, and the sensor, wherein the multiplexor is configured to multiplex the first EM wave and the second EM wave.

3. The device of claim 1, wherein the at least a second waveguide comprises a ring resonator.

4. The device of claim 1, wherein the at least a first waveguide is configured to provide communication between the first EM wave and a portion of the sample by propagating an evanescent wave from a surface.

5. The device of claim 1, wherein the first optical property includes an index of refraction of the at least a first waveguide.

6. The device of claim 1, wherein the at least a first waveguide is configured to provide communication between the first EM wave and a portion of the sample by propagating surface plasmons upon a surface.

7. The device of claim 1, wherein a first surface is coated with a capture antibody configured to selectively bind to a first antigen and vary the first optical property.

8. The device of claim 1, wherein the microfluid device is configured to contain a first marker, wherein the first marker is configured to selectively conjugate the first antigen and vary the first optical property.

9. The device of claim 1, further comprising:
A computing device in communication with the sensor and configured to:
receive, from the at least a sensor, at least a signal representing the at least a first constituent and the at least a second constituent; and
communicate, with a remote device, at least a first datum representing the at least a first constituent and at least a second datum representing the at least a second constituent.

10. The device of claim 1, wherein the remote device is configured to process the at least a first datum and the at least a second datum; and
recognize a variant of the substance, as a function of processing the at least a first datum and the at least a second datum.

11. A method of detection, the method comprising:
receiving, by an input of a housing, a sample;
propagating, by at least a first waveguide in fluidic communication with the input, a first electromagnetic (EM) wave;
varying, by the at least a first waveguide, in a first optical property as a function of the sample;
propagating, by at least a second waveguide in fluidic communication with the input, a second electromagnetic (EM) wave;
varying, by the at least a second waveguide, in a second optical property as a function of the sample;
detecting, by at least a sensor in communication with the at least a first waveguide and the at least a second waveguide, using the first EM wave and the second EM wave, a first constituent of the sample and a second constituent of the sample, as a function of a first variance in the first optical property and a second variance in the second optical property.

12. The method of claim 11, further comprising a multiplexor in communication with the at least a first waveguide, the at least a second waveguide, and the sensor, wherein the multiplexor is configured to multiplex the first EM wave and the second EM wave.

13. The method of claim 11, wherein the at least a second waveguide comprises a ring resonator.

14. The method of claim 11, wherein the at least a first waveguide is configured to provide communication between the first EM wave and a portion of the sample by propagating an evanescent wave from a surface.

15. The method of claim 11, wherein the first optical property includes an index of refraction of the at least a first waveguide.

16. The method of claim 11, wherein the at least a first waveguide is configured to provide communication between the first EM wave and a portion of the sample by propagating surface plasmons upon a surface.

17. The method of claim 11, wherein a first surface is coated with a capture antibody configured to selectively bind to a first antigen and vary the first optical property.

18. The method of claim 11, wherein the microfluid device is configured to contain a first marker, wherein the first marker is configured to selectively conjugate the first antigen and vary the first optical property.

19. The method of claim 11, further comprising:
a computing device in communication with the sensor and configured to:
receive, from the at least a sensor, at least a signal representing the at least a first constituent and the at least a second constituent; and
communicate, with a remote device, at least a first datum representing the at least a first constituent and at least a second datum representing the at least a second constituent.

20. The method of claim 11, wherein the remote device is configured to process the at least a first datum and the at least a second datum; and
recognize a variant of the substance, as a function of processing the at least a first datum and the at least a second datum.
